(19) United States
(12) Patent Application Publication
(10) Pub. No.: US 2020/0375919 A1
(43) Pub. Date: Dec. 3, 2020

(54) BONE GRAFT MATERIAL WITH 4-HEXYLRESORCINOL THAT AFFECTS BONE FORMATION

(71) Applicants: MANTIZ LOGITECH CO., LTD., Daegu (KR); BONEGENE.co.,Ltd., Daegu (KR)

(72) Inventors: Dae Chul CHO, Daegu (KR); Je Yong CHOI, Daegu (KR); Seong Hwan KIM, Daegu (KR); Ki Young KIM, Daegu (KR)

(73) Assignees: MANTIZ LOGITECH CO., LTD., Daegu (KR); BONEGENE.co.,Ltd., Daegu (KR)

(21) Appl. No.: 16/884,300

(22) Filed: May 27, 2020

(30) Foreign Application Priority Data
May 29, 2019 (KR) 10-2019-0062864

(51) Int. Cl. A61K 31/05; A61K 47/38; A61K 47/42; A61K 47/36; A61K 47/02; A61P 19/00

(52) U.S. Cl. CPC A61K 31/05; A61K 47/38; A61P 19/00; A61K 47/36; A61K 47/02; A61K 47/42

(57) ABSTRACT

The present disclosure relates to a bone graft material grafted with 4-hexylresorcinol affecting bone formation, the bone graft material which not only can rapidly promote bone formation by mixing hydrous ethanol having low concentration 4-hexylresorcinol dissolved therein and a base material for release control with distilled water or a salt-dissolved aqueous solution to obtain a mixed solution and precipitating a bone graft material into the mixed solution, thereby injecting 4-hexylresorcinol into the bone graft material, but also can have a consistent treatment effect by adjusting elution amount of 4-hexylresorcinol through the base material for release control, thereby allowing 4-hexylresorcinol to be slowly released during a treatment period.

### CROSS-REFERENCE TO RELATED APPLICATIONS

A claim for priority under 35 U.S.C. § 119 is made to Korean Patent Application No. 10-2019-0062864 filed May 29, 2019 in the Korean Intellectual Property Office, the entire contents of which are hereby incorporated by reference.

### BACKGROUND OF THE INVENTION

### Field of the Invention

The present invention relates to a bone graft material grafted with 4-hexylresorcinol affecting bone formation, the bone graft material capable of promoting bone formation by using a bone graft material having 4-hexylresorcinol added thereto.

### Related Art

When performing a disc surgery, an empty space is filled using autogenous bone or allogenic bone after performing a cage inserting process. This is the most preferable method considering an immune reaction and various side effects. However, effects may vary depending on the condition of a patient and the condition of allogenic bone, and there may not be the effects when using a bone which has already lost the ability of cells. Further, there are limitations in acquisition of the autogenous bone and allogenic bone.

For this reason, we can expect a speedy recovery by making a synthetic bone graft material using material which improves the ability of bone forming cells that have already been inside the body. Of course, a bone graft material to which BMP-2 giving a good effect to bone formation is added has already been developed. However, the bone graft material has not shown a good result as problems including a cost problem and a fatal side effect exist in the bone graft material. There have been techniques for producing a bone graft material using 4-hexylresorcinol so as to overcome such problems. Korean Registered Patent No. 10-1667245, “Technique of injecting 4-hexylresorcinol into graft material using interaction between hydrophobic materials”, comprises the steps of preparing a mixed solution by mixing anhydrous ethanol having 4-hexylresorcinol dissolved therein or hydrous ethanol containing not more than 10 vol % of water with distilled water or an aqueous solution having a salt dissolved therein; and precipitating a bone graft material in the mixed solution prepared in the step of preparing the mixed solution to inject 4-hexylresorcinol into the bone graft material by hydrophobic interaction.

Although Korean Registered Patent No. 10-1667245 relates to a method for penetrating 4-hexylresorcinol into a graft material at a high concentration, cell viability is excellent at low concentrations rather than high concentrations, and osteoplastic facilitation has also been observed to be higher at the low concentrations as various experimental results.

Further, although Korean Registered Patent No. 10-1667245 focuses on contents which reduce foreign body reaction of a bone graft material in a human body and can obtain an antibacterial effect in the human body, a method for promoting formation of osteoblasts, inhibiting formation of osteoclasts, and allowing 4-hexylresorcinol to be slowly released during a treatment period, thereby enabling a consistent effect to be obtained is necessary since the reason of using 4-hexylresorcinol is that 4-hexylresorcinol acts as an aid for improving the ability of the bone graft material.

### RELATED ART DOCUMENT

### Patent Document

(Patent document 1) Korean Registered Patent No. 10-1667245, Technique of injecting 4-hexylresorcinol into graft material using interaction between hydrophobic materials

### SUMMARY OF THE INVENTION

In order to solve the above-mentioned problems, the purpose of the present invention is to provide a bone graft material grafted with 4-hexylresorcinol affecting bone formation, the bone graft material which not only can rapidly promote bone formation by mixing hydrous ethanol having low concentration 4-hexylresorcinol dissolved therein and a base material for release control with distilled water or a salt-dissolved aqueous solution to obtain a mixed solution and precipitating a bone graft material into the mixed solution, thereby injecting 4-hexylresorcinol into the bone graft material, but also can have a consistent treatment effect by adjusting elution amount of 4-hexylresorcinol through the base material for release control, thereby allowing 4-hexylresorcinol to be slowly released during a treatment period.

A bone graft material grafted with 4-hexylresorcinol affecting bone formation according to the present invention for achieving the above-mentioned purpose is produced by mixing hydrous ethanol containing not more than 60 vol % of water having 4-hexylresorcinol dissolved therein and a base material for release control with distilled water or a salt-dissolved aqueous solution to prepare a mixed solution and precipitating a bone graft material into the mixed solution, thereby injecting 4-hexylresorcinol into the bone graft material.

Further, a bone graft material grafted with 4-hexylresorcinol affecting bone formation according to the present invention is characterized in that 0.001 to 0.5 part by weight of 4-hexylresorcinol and 0.003 to 0.7 part by weight of the bone graft material are mixed with respect to 100 parts by weight of the hydrous ethanol containing not more than 60 vol % of water.

Further, the bone graft material includes one or more selected from hydroxyapatite, tricalcium phosphate, monocalcium phosphate, tetracalcium phosphate, and dicalcium phosphate.

Further, the base material for release control includes one or more selected from hydroxypropyl methylcellulose (HPMC), methyl cellulose (MC), hydroxyethyl methyl cellulose (HEMC), ethyl cellulose (EC), carboxymethyl cellulose (CMC), collagen, and hyaluronic acid, and 5 to 20 parts by weight of the base material for release control is mixed with respect to 100 parts by weight of the hydrous ethanol containing not more than 60 vol % of water.

A bone graft material grafted with 4-hexylresorcinol affecting bone formation according to the present invention further promotes differentiation of osteoblasts by grafting low concentration 4-hexylresorcinol instead of conventional high concentration 4-hexylresorcinol with a bone graft material, and enables the bone graft material which has performed only the role of a filling material after spinal surgery to perform the role of a therapeutic agent inducing rapid bone formation by effectively inhibiting differentiation of osteoblasts. Further, a bone graft material grafted with 4-hexylresorcinol affecting bone formation according to the present invention not only can be produced at low costs, but also can minimize side effects.

Further, a bone graft material grafted with 4-hexylresorcinol affecting bone formation according to the present invention may have a consistent effect by additionally mixing a base material for release control to adjust elution amount of 4-hexylresorcinol, thereby allowing 4-hexylresorcinol to be slowly released during a treatment period.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing results of an MTT Assay (a cellular proliferative response measuring method) experiment made on each of a bone graft material grafted with low concentration 4-hexylresorcinol according to the present invention, a bone graft material which does not use 4-hexylresorcinol, and a bone graft material which uses high concentration 4-hexylresorcinol.

FIG. 2 is a graph showing results of an Alkaline Phosphatase Staining (an early-stage osteoblastic differentiation confirming) experiment made on each of the bone graft material grafted with low concentration 4-hexylresorcinol according to the present invention, the bone graft material which does not use 4-hexylresorcinol, and the bone graft material which uses high concentration 4-hexylresorcinol.

FIG. 3 is a graph showing results of an Alizarin Red S Staining (a later-stage osteoblastic differentiation confirming) experiment made on each of the bone graft material grafted with low concentration 4-hexylresorcinol according to the present invention, the bone graft material which does not use 4-hexylresorcinol, and the bone graft material which uses high concentration 4-hexylresorcinol.

FIGS. 4A to 4D are graphs showing results of an mRNA (Messenger RiboNucleic Acid) expression amount experiment made on each of the bone graft material grafted with low concentration 4-hexylresorcinol according to the present invention, the bone graft material which does not use 4-hexylresorcinol, and the bone graft material which uses high concentration 4-hexylresorcinol.

FIG. 5 is results of confirming a protein level through a western blot with respect to each of the bone graft material grafted with low concentration 4-hexylresorcinol according to the present invention, the bone graft material which does not use 4-hexylresorcinol, and the bone graft material which uses high concentration 4-hexylresorcinol.

FIGS. 6A and 6B are results of experimenting differentiation of osteoclasts with respect to each of the bone graft material grafted with low concentration 4-hexylresorcinol according to the present invention, the bone graft material which does not use 4-hexylresorcinol, and the bone graft material which uses high concentration 4-hexylresorcinol.

FIG. 7 is a graph showing results of viscosity experiments made on various Examples and Comparative Examples grafted with low concentration 4-hexylresorcinol according to the present invention.

FIG. 8 is a graph showing results of elution rate experiments made on various Examples and Comparative Examples grafted with low concentration 4-hexylresorcinol according to the present invention.

FIG. 9 is a graph showing results of moisture content experiments made on various Examples and Comparative Examples grafted with low concentration 4-hexylresorcinol according to the present invention.

### DESCRIPTION OF EXEMPLARY EMBODIMENTS

Advantages and characteristics of the present invention, and a method for achieving them will be obvious if referring to embodiments described later in detail together with the accompanying drawings.

However, the present invention is not limited to the embodiments disclosed below, but will be implemented in various forms which are different from one another.

These embodiments in the present specification may be provided to completely disclose the present invention and allow those skilled in the art to which the present invention pertains to completely know the scope of the present invention.

The present invention will only be defined by the appended claims.

Therefore, well-known elements, well-known operations and well-known techniques in some embodiments will not be described specifically to avoid the ambiguous interpretation of the present invention.

Further, the same elements will be denoted by the same reference numerals throughout the specification, and terms used (mentioned) in the present specification are for explaining the embodiments rather than limiting the present invention.

In the specification, a single type includes even a plural type as long as not particularly mentioned, and an element and an operation mentioned as “include (or be furnished with)” do not exclude existence or addition of one or more other elements and operations.

Unless otherwise defined, all terms (including technical and scientific terms) used in the present specification could be used as meanings commonly understood by those ordinary skilled in the art to which the present invention pertains.

Further, unless defined otherwise, terms defined in generally used dictionaries are not interpreted ideally or excessively.

First, material constituting the present invention will be described as follows.

4-hexylresorcinol is an organic compound having various activities of bacterial origin. 4-hexylresorcinol had been widely used as an antiseptic using antimicrobial action of 4-hexylresorcinol before 1970. It has recently been reported that 4-hexylresorcinol inhibits activities of the cancer cells by inhibiting NF-kB pathway in cancer cells. Since NF-kB pathway contributes to activities of osteoclasts also, 4-hexylresorcinol can increase bone density by inhibiting the activities of osteoclasts. Further, 4-hexylresorcinol can inhibit formation of foreign body reaction giant cells by inhibiting diacylglycerol kinase.

Although hydroxyapatite having an abbreviated name of HA is a mineral which has a formula Ca₅(PO₄)₃(OH) and is generated from the nature, hydroxyapatite is usually represented by Ca₁₀(PO₄)₆(OH)₂ to express that a crystal unit cell of the mineral is formed of two entities. A pure hydroxyapatite powder is white. However, hydroxyapatite generated from the nature may be brown, yellow or green as much as it is comparable with dental fluorosis. Hydroxyapatite having carbonized calcium coming out thereof is a main material which forms tooth enamel and dentine.

Hydroxypropyl methylcellulose (HPMC) is a semisynthetic agent which is used as an additive for foods, drugs and cosmetic products, and others.

As the name suggests, hydroxypropyl methylcellulose (HPMC) is a cellulose derivative. HPMC has a methoxy group (—OCH₃), a hydroxypropoxy group (—OCH₂CH(CH₃)OH), or hydrogen as a substituent. HPMC is used as an emulsifier, a thickening stabilizer, a suspending agent, a moisturizer, etc. HPMC is used also in foods, cosmetic products, coating agents, etc. When HPMC is used in preparations, HPMC may be used in preparing an ophthalmic preparation or preparing a controlled release formulation for oral administration such as a tablet with sustained release, a tablet with rapid release or the like using physicochemical characteristics of HPMC. HPMC may be used for increasing strength of tablets. HPMC is formed in the form of a white or yellow powder or granule. HPMC is hardly dissolved in anhydrous ethanol, and a solution of which volume is increased, and which has a slightly turbid consistency is prepared when HPMC is put into water. Volume-increasing and sticky properties possessed by HPMC when putting HPMC into water are properties which are important in preparing a controlled release formulation for oral administration. If a large amount of HPMC is used when covering the outer surface of a drug with HPMC, a concentration gradient of the drug is decreased, and a release velocity of the drug is lowered as distance of moving the drug through diffusion increases. Due to volume-increasing properties of HPMC when HPMC is used in a very small amount, a tablet with rapid release may be prepared from HPMC. HPMC as a cellulose derivative also has hygroscopicity.

Hereinafter, preferred embodiments of the present invention will now be described in detail with reference to the accompanying drawings.

4-hexylresorcinol (4HR) is a functional material (bioactive material) which can be used by mixing 4-hexylresorcinol with a bone graft material, and a bone graft material having 4-hexylresorcinol added thereto increases activities of alkaline phosphatase in osteoblasts, increases adhesive force and adhesion speed of osteoblasts on the surface of the bone graft material, and can significantly improve adhesion with bones.

The term “bone graft material” used in the present invention means a graft material which is used to fill a space within the osseous tissue by replacing the defect part and promote the formation of new bones when a defect part is generated in osseous tissue by various diseases.

Since 4-hexylresorcinol according to the present invention used as a functional material is not well dissolved in water, ethanol is used as 4-hexylresorcinol. Ethanol has widely been used as a solvent in the production of biomaterial. However, since hydroxypropyl methylcellulose (HPMC) is hardly dissolved in anhydrous ethanol, hydrous ethanol is used.

A bone graft material grafted with 4-hexylresorcinol affecting bone formation according to the present invention is produced by a method of mixing hydrous ethanol containing not more than 60 vol % of water having 4-hexylresorcinol dissolved therein and a base material for release control with distilled water or a salt-dissolved aqueous solution to prepare a mixed solution and precipitating a bone graft material into the mixed solution, thereby injecting 4-hexylresorcinol into the bone graft material.

A method of producing a bone graft material according to the present invention comprises the step of mixing hydrous ethanol containing not more than 60 vol % of water having 4-hexylresorcinol dissolved therein and a base material for release control with a solvent (distilled water or a salt-dissolved aqueous solution). Through such a step, hydrophobic alkyl moiety of 4-hexylresorcinol is agglomerated inward to form an emulsion, and then 4-hexylresorcinol may be injected into a graft material at low concentration through hydrophobic interaction. At this time, the salt may include calcium salts or magnesium salts.

If 4-hexylresorcinol is dissolved in ethanol in the present invention, the ethanol is not particularly limited, and the ethanol may include hydrous ethanol containing not more than 60 vol % of water. Since it is important to mix 4-hexylresorcinol at a low concentration in the present invention, it is difficult to use hydrous ethanol containing not more than 10 vol % of water.

However, it is difficult to penetrate the 4-hexylresorcinol into the graft material since dissolution rate is lowered although 4-hexylresorcinol has a low concentration if hydrous ethanol contains more than 60 vol % of water.

Further, since a base material for release control is rarely dissolved in anhydrous ethanol, it is important to use hydrous ethanol containing not less than 10 vol % to not more than 60 vol % of water.

Specifically, a bone graft material grafted with 4-hexylresorcinol affecting bone formation according to the present invention may be produced by mixing 0.001 to 0.5 part by weight of 4-hexylresorcinol and 0.003 to 0.7 part by weight of a bone graft material with respect to 100 parts by weight of hydrous ethanol containing not more than 60 vol % of water. Here, it is important to mix 4-hexylresorcinol and the bone graft material to low concentrations. It has been confirmed that a bone graft material grafted with 4-hexylresorcinol affecting bone formation according to the present invention as a bone graft material has excellent performance as results of cell experiments when 4-hexylresorcinol is mixed at low concentrations compared to when 4-hexylresorcinol is mixed at high concentrations.

This will be described below in detail with reference to the drawings.

The bone graft material may comprise one or more selected from hydroxyapatite, tricalcium phosphate, monocalcium phosphate, tetracalcium phosphate, and dicalcium phosphate.

Further, the base material for release control includes one or more selected from hydroxypropyl methylcellulose (HPMC), methyl cellulose (MC), hydroxyethyl methyl cellulose (HEMC), ethyl cellulose (EC), carboxymethyl cellulose (CMC), collagen, and hyaluronic acid, and a mixed solution is prepared by mixing 5 to 20 parts by weight of the base material for release control with respect to 100 parts by weight of the hydrous ethanol containing not more than 60 vol % of water.

A bone graft material according to the present invention may be used in the form of a putty, a paste, a moldable strip, a block, a chip or the like obtained by performing a molding process using a method of pressing, compression, pressurizing contact, packing, pressure, solidifying or the like. A bone graft material according to the present invention may be used in the form of a formulation such as a gel, a powder, a paste, a tablet, a pellet or the like obtained by using chemical additives, or may be used in the form of the powder itself.

In the present invention, a bone graft material comprising 4-hexylresorcinol is produced by reacting the mixed solution prepared by aforementioned method with the bone graft material.

At this time, the reaction process may be performed by a method of precipitating the bone graft material in the mixed solution, and 4-hexylresorcinol may be penetrated into the bone graft material by hydrophobic interaction.

### [Experimental Example] Comparative Experiments of High and Low Concentration 4-Hexylresorcinol

Concentration 0.0194% (=10⁻⁴M) and concentration 3% of 4-hexylresorcinol with regard to the differentiation of cells were comparatively evaluated through various experiments.

MTS Measuring Method of Cytotoxicity and Cell Viability

Mitochondrial NADH-dehydrogenase forms formazan having a color by reducing a tetrazolium salt such as MTS. Therefore, as the number of cells which express dehydrogenase, i.e., living cells is proportional to formation of formazan, the living cells can be quantified.

After seeding human primary cells, adhering the seeded-human primary cells to a cell plate, and treating the seeded-human primary cells adhered to the cell plate with an MTS Cell Proliferation Assay Kit (ab197010, UK, abcam) every 24 hours and 48 hours, resulting materials were confirmed by using a wavelength of 490 nm.

Method of Measuring Cell Culture and Alkaline Phosphatase Activities

In vitro tests were performed by using osteoblast-like cells through a human primary cell culture. A cell survival environment was progressed by supplementing DMEM (Dulbecco Modified Eagle's Medium)-high glucose (PAA Laboratories, Pasching, Austria) and 20% fetal bovine serum (PAA, Canada) and comprising 1% penicillin/streptomycin (100×), and media were exchanged every other day. Resulting materials were confirmed on the seventh day of differentiation experiments by using alkaline phosphatase (Sigma, St. Louis, Mo., USA).

Method of Measuring Bone Differentiation Degrees by Dyeing Calcium Accumulation of Bone-Differentiated Cells (Alizarin Red S Staining)

In vitro tests were performed by using osteoblast-like cells through a human primary cell culture. A cell survival environment was progressed by supplementing DMEM (Dulbecco Modified Eagle's Medium)-high glucose (PAA Laboratories, Pasching, Austria) and 20% fetal bovine serum (PAA, Canada) and comprising 1% penicillin/streptomycin (100×), and media were exchanged every other day. Resulting materials were confirmed on the seventh day of differentiation experiments.

Method of Measuring Differentiation Abilities of Osteoclasts (TRAP Staining)

In vitro tests were performed by using osteoclasts through a human bone marrow derived macrophages culture. A cell survival environment was progressed by supplementing DMEM (Dulbecco Modified Eagle's Medium)-high glucose (PAA Laboratories, Pasching, Austria) and 20% fetal bovine serum (PAA, Canada) and comprising 1% penicillin/streptomycin (100×), and media were exchanged once a day. Resulting materials were confirmed on the fifth day of differentiation experiments through TRAP staining.

### Experimental Results

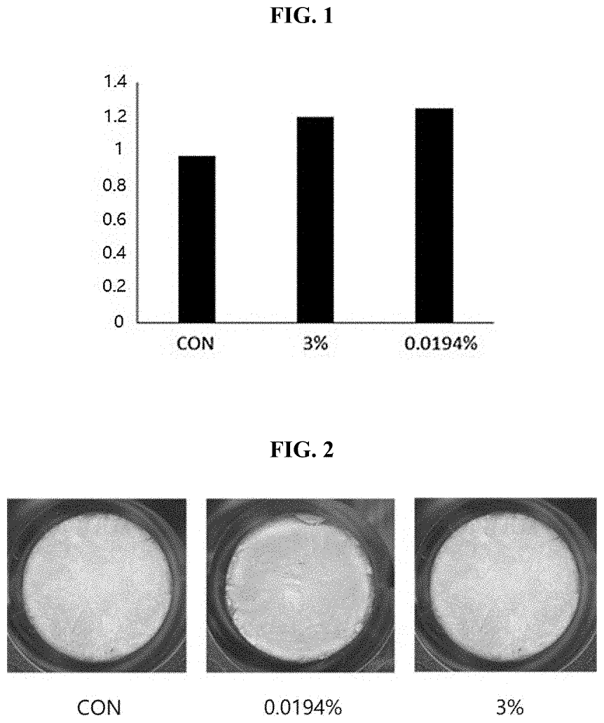

By referring to FIG. 1 that cell viabilities are higher than a control (4-hexylresorcinol X) as results of toxicity test which has been proceeded at 4-hexylresorcinol concentrations of 3% and 0.0194% (=10⁻⁴M), it can be seen that 4-hexylresorcinol has an effect of increasing cell proliferation, and it is confirmed that cell proliferation is more active when 4-hexylresorcinol has a low concentration of 0.0194% rather than a high concentration of 3%.

Referring to FIG. 2, it is observed that 4-hexylresorcinol with a low concentration of 0.0194% (=10⁻⁴M) has a high ALP activity compared to 4-hexylresorcinol with a high concentration of 3% and control as results of proceeding with an ALP test, i.e., an early differentiation experiment of osteoblasts.

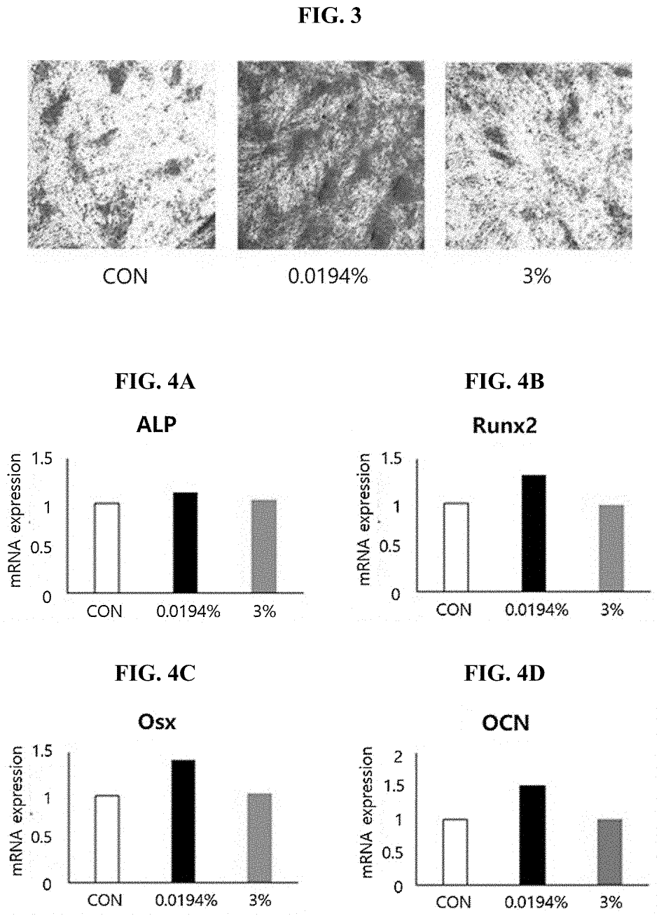

Referring to FIG. 3, it is observed that 4-hexylresorcinol with a low concentration of 0.0194% (=10⁻⁴M) determined through concentration screening has a high calcification compared to 4-hexylresorcinol with a high concentration of 3% and control by proceeding Alizarin Red S staining test, i.e., a late differentiation experiment of osteoblasts.

Referring to FIGS. 4A to 4D, it can be observed that treating 4-hexylresorcinol with a low concentration of 0.0194% (=10⁻⁴M) has a larger expression level than treating 4-hexylresorcinol with a high concentration of 3% or treating control even when checking an mRNA expression level through an early differentiation marker (ALP) and a late differentiation marker (Runx2, Osx, Ocn) of osteoblasts.

Referring to FIG. 5, it can be confirmed that treating 4-hexylresorcinol with a low concentration of 0.0194% has ALP and Runx2 expression levels having effects on differentiation of osteoblasts, the ALP and Runx2 expression levels which are higher than treating 4-hexylresorcinol with a high concentration of 3% or treating control even when checking protein levels according to the checked mRNA levels through a western blot after checking mRNA levels.

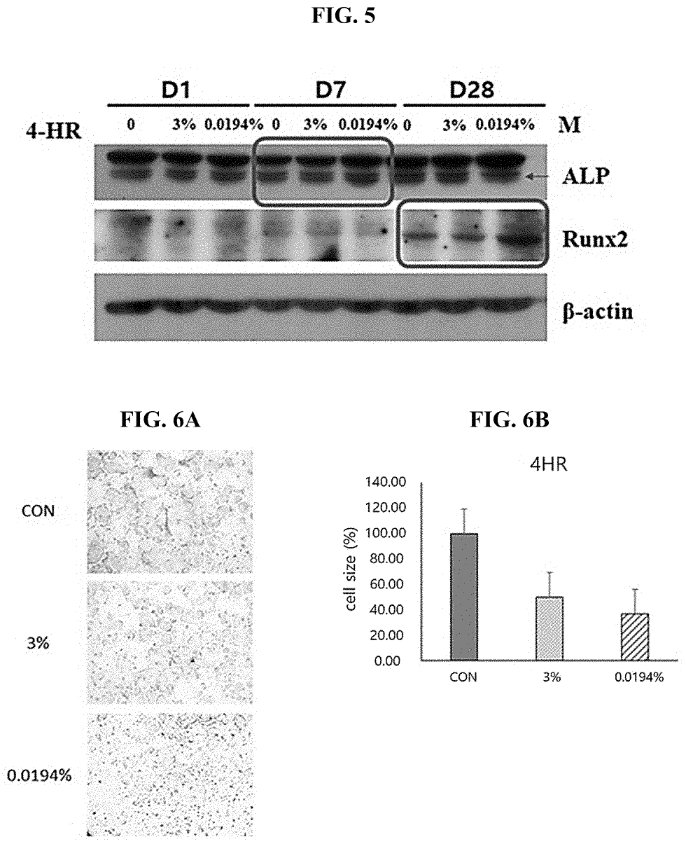

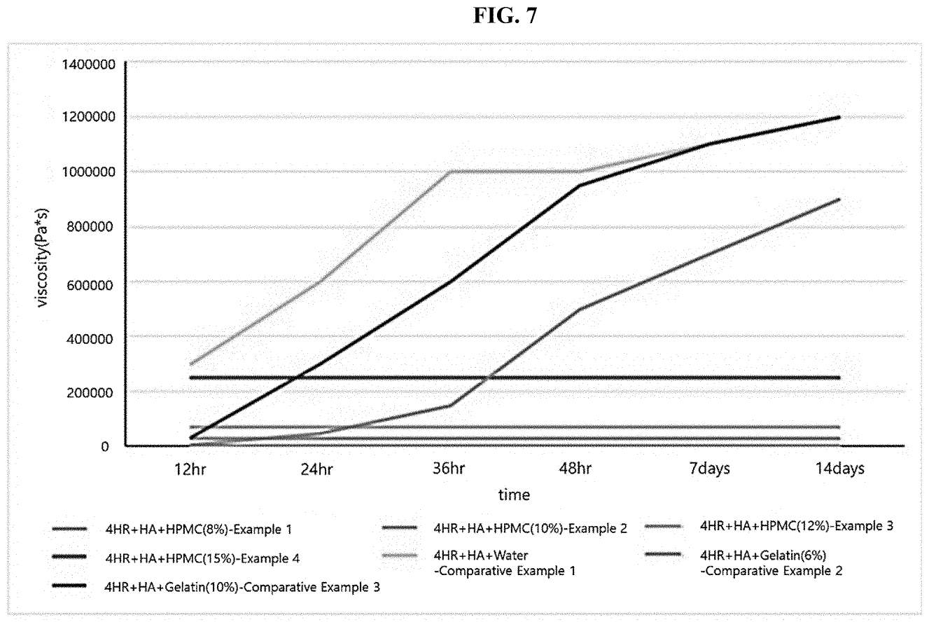

Referring to FIGS. 6A and 6B, it can be confirmed that differentiation of the osteoclasts is inhibited by treating 4-hexylresorcinol even when examining differentiation of osteoclasts affecting bone formation through bone resorption. It is confirmed that an effect of the differentiation of the osteoclasts is more excellent when treating 4-hexylresorcinol with a low concentration of 0.0194% than when treating 4-hexylresorcinol with a high concentration of 3%. It can be seen through these data that 4-hexylresorcinol promotes bone formation by inhibiting the differentiation of the osteoclasts.

When summarizing the various experimental results described above, it can be seen that treating 4-hexylresorcinol with a low concentration of 0.0194% (=10⁴M) according to the present invention is more effective in bone formation than treating 4-hexylresorcinol with a high concentration of 3% according to a conventional technique or treating control.

### EXAMPLES AND COMPARATIVE EXAMPLES

In all of the following Examples and Comparative Examples, tests were performed by using 4-hexylresorcinol (4HR) with a low concentration of 0.0194% (=10⁻⁴ M) and varying concentration of hydroxypropyl methylcellulose (HPMC).

### [Example 1] Preparing a Bone Graft Material by Mixing 4HR (0.0194%), Hydroxyapatite (HA), and HPMC (8%)

0.00194 g of 4-hexylresorcinol (4HR) was dissolved in 10 ml of hydrous ethanol containing 50 vol % of water to obtain a 4HR-dissolved ethanol. A mixed solution was prepared by mixing the 4HR-dissolved ethanol and 0.8 g of HPMC as a base material for release control with distilled water, thereby diluting the 4HR-dissolved ethanol and base material for release control with distilled water at a ratio of 1:10.

After putting 0.006 g of a hydroxyapatite flake (disc) with a diameter of 8 mm into the mixed solution, precipitating the hydroxyapatite flake in the mixed solution for 10 minutes, and taking the hydroxyapatite flake out of the mixed solution, a resulting material was dried in a dryer of 50° C. for 10 hours.

### [Example 2] Preparing a Bone Graft Material by Mixing 4HR (0.0194%), Hydroxyapatite (HA), and HPMC (10%)

A bone graft material was produced by the same method as in Example 1 except that 1 g of HPMC was mixed.

### [Example 3] Preparing a Bone Graft Material by Mixing 4HR (0.0194%), Hydroxyapatite (HA), and HPMC (12%)

A bone graft material was produced by the same method as in Example 1 except that 1.2 g of HPMC was mixed.

### [Example 4] Preparing a Bone Graft Material by Mixing 4HR (0.0194%), Hydroxyapatite (HA), and HPMC (15%)

A bone graft material was produced by the same method as in Example 1 except that 1.5 g of HPMC was mixed.

### [Comparative Example 1] Preparing a Bone Graft Material by Mixing 4HR (0.0194%), Hydroxyapatite (HA), and Water

0.00194 g of 4-hexylresorcinol (4HR) was dissolved in 10 ml of hydrous ethanol containing 50 vol % of water to obtain a 4HR-dissolved ethanol. A mixed solution was prepared by mixing the 4HR-dissolved ethanol with distilled water, thereby diluting the 4HR-dissolved ethanol with distilled water at a ratio of 1:10.

After putting 0.006 g of a hydroxyapatite flake (disc) with a diameter of 8 mm into the mixed solution, precipitating the hydroxyapatite flake in the mixed solution for 10 minutes, and taking the hydroxyapatite flake out of the mixed solution, a resulting material was dried in a dryer of 50° C. for 10 hours.

### [Comparative Example 2] Preparing a Bone Graft Material by Mixing 4HR (0.0194%), Hydroxyapatite (HA), and Gelatin (6%)

0.00194 g of 4-hexylresorcinol (4HR) was dissolved in 10 ml of hydrous ethanol containing 50 vol % of water to obtain a 4HR-dissolved ethanol. A mixed solution was prepared by mixing the 4HR-dissolved ethanol and 0.6 g of gelatin as a base material for release control with distilled water, thereby diluting the 4HR-dissolved ethanol and base material for release control with distilled water at a ratio of 1:10.

After putting 0.006 g of a hydroxyapatite flake (disc) with a diameter of 8 mm into the mixed solution, precipitating the hydroxyapatite flake in the mixed solution for 10 minutes, and taking the hydroxyapatite flake out of the mixed solution, a resulting material was dried in a dryer of 50° C. for 10 hours.

### [Comparative Example 3] Preparing a Bone Graft Material by Mixing 4HR (0.0194%), Hydroxyapatite (HA), and Gelatin (10%)

A bone graft material was produced by the same method as in Comparative Example except that 1 g of gelatin was mixed.

Referring to Table and FIG. 7, it can be seen that viscosities in Examples 1 to 4 are constant even when time goes by. It is confirmed from Examples 1 to 4 that the higher the contents of HPMC are, i.e., when the contents of HPMC are 15%, the highest levels the viscosities constantly maintain for a long time.

Namely, viscosity effects may be constantly maintained for a long time by additionally mixing hydroxypropyl methylcellulose (HPMC). This is associated with elution rates of 4-hexylresorcinol which will be examined below.

TABLE 1
Viscosity comparison

| Viscosity | 12 hr | 24 hr | 36 hr | 48 hr | 7 days | 14 days |
|---|---|---|---|---|---|---|
| 4HR + HA + HPMC(8%)-Example 1 | 3000 | 2990 | 2980 | 2975 | 2950 | 2920 |
| 4HR + HA + HPMC(10%)-Example 2 | 30000 | 29950 | 29940 | 29935 | 29930 | 29920 |
| 4HR + HA + HPMC(12%)-Example 3 | 70000 | 69990 | 69980 | 69975 | 69970 | 69950 |
| 4HR + HA + HPMC(15%)-Example 4 | 250000 | 249950 | 249945 | 249930 | 249925 | 249920 |
| 4HR + HA + water-Comparative | 300000 | 600000 | 1000000 | 1000000 | 1100000 | 1200000 |
| Example 1 | | | | | | |
| 4HR + HA + gelatin(6%)-Comparative | 5000 | 50000 | 150000 | 500000 | 700000 | 900000 |
| Example 2 | | | | | | |
| 4HR + HA + gelatin(10%)-Comparative | 35000 | 300000 | 600000 | 950000 | 1100000 | 1200000 |
| Example 3 | | | | | | |

However, it is confirmed that viscosities are radically increased in bone graft materials of Comparative Examples 1 to 3 in which hydroxypropyl methylcellulose (HPMC) has not been mixed as time goes by. A consistent effect may be obtained by constantly maintaining the viscosities without radically increasing the viscosities, thereby allowing 4-hexylresorcinol to be slowly released during a treatment period.

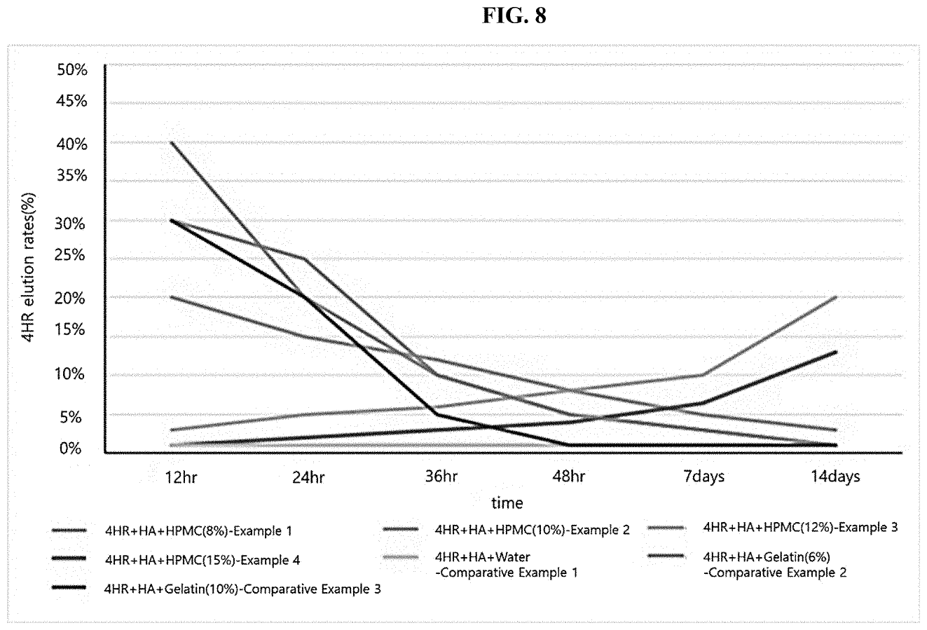

Referring to Table 2 and FIG. 8, although bone graft material of Example 1 with an HPMC content of 8% initially elutes a large amount of 4-hexylresorcinol similarly as in bone graft materials of Comparative Examples which do not contain HPMC, an elution amount of the bone graft material of Example 1 with an HPMC content of 8 is radically decreased as time goes by. This bone graft material has a problem that a consistent treatment effect by 4-hexylresorcinol during a treatment period cannot be obtained.

TABLE 2
Comparison of 4-hexylresorcinol elution rates

| 4HR elution rate | 12 hr | 24 hr | 36 hr | 48 hr | 7 days | 14 days |
|---|---|---|---|---|---|---|
| 4HR + HA + | 30% | 25% | 10% | 5% | 3% | 1% |
| HPMC(8%)-Example 1 | | | | | | |
| 4HR + HA + | 20% | 15% | 12% | 8% | 5% | 3% |
| HPMC(10%)-Example 2 | | | | | | |
| 4HR + HA + | 3% | 5% | 6% | 8% | 10% | 20% |
| HPMC(12%)-Example 3 | | | | | | |
| 4HR + HA + | 1% | 2% | 3% | 4% | 7% | 13% |
| HPMC(15%)-Example 4 | | | | | | |
| 4HR + HA + | 1% | 1% | 1% | 1% | 1% | 1% |
| water-Comparative | | | | | | |
| Example 1 | | | | | | |
| 4HR + HA + | 40% | 20% | 10% | 5% | 3% | 1% |
| gelatin(6%)- | | | | | | |
| Comparative | | | | | | |
| Example 2 | | | | | | |
| 4HR + HA + | 30% | 20% | 5% | 1% | 1% | 1% |
| gelatin(10%)- | | | | | | |
| Comparative | | | | | | |
| Example 3 | | | | | | |

Although a bone graft material of Example 2 is good compared to that of Example 1, a 4-hexylresorcinol elution amount of the bone graft material of Example 2 is slowly decreased as time goes by.

It is confirmed that, as time goes by, bone graft materials of Examples 3 and 4 have increased elution amounts of 4-hexylresorcinol, and, as 4-hexylresorcinol is slowly increased in the bone graft material of Example 4 compared to that of Example 3, the bone graft material of Example 4 is suitable for obtaining consistent treatment effect due to 4-hexyresorcinol.

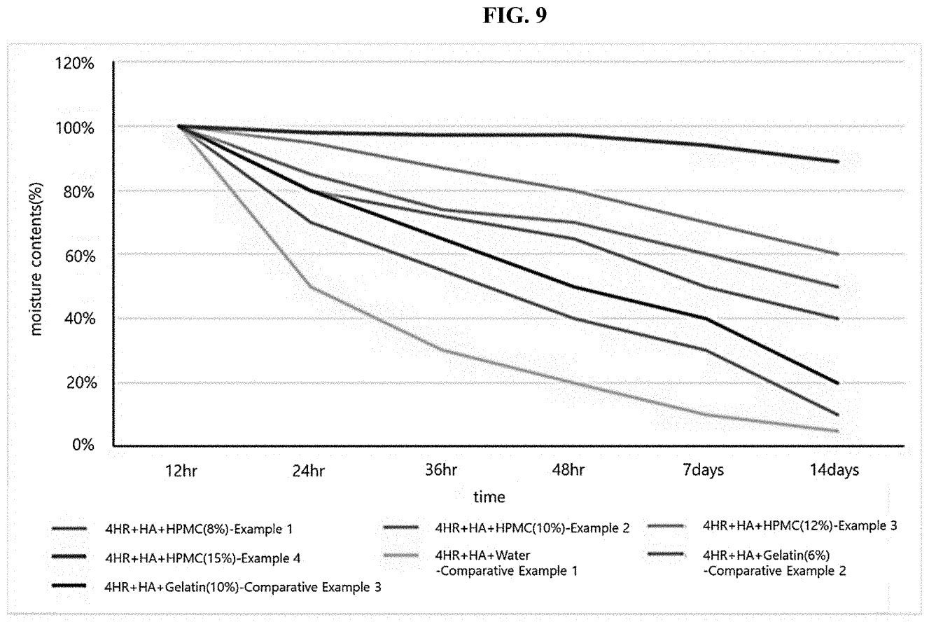

Referring to Table and FIG. 9, it is confirmed that the bone graft material of Example 4 is the most suitable for expecting a consistent treatment effect due to 4-hexyresorcinol since, although time goes by, a moisture content is not greatly changed, but is slowly decreased in the bone graft material of Example 4 corresponding to an HPMC content of 15% among the bone graft materials of Examples 1 to 4. Since there is problem in elution of 4-hexylresorcinol when moisture is removed, it is very important to constantly maintain the moisture for along time.

TABLE 3
Comparison of moisture contents

| Moisture content | 12 hr | 24 hr | 36 hr | 48 hr | 7 days | 14 days |
|---|---|---|---|---|---|---|
| 4HR + HA + | 100% | 80% | 72% | 65% | 50% | 40% |
| HPMC(8%)-Example 1 | | | | | | |
| 4HR + HA + | 100% | 85% | 74% | 70% | 60% | 50% |
| HPMC(10%)-Example 2 | | | | | | |
| 4HR + HA + | 100% | 95% | 87% | 80% | 70% | 60% |
| HPMC(12%)-Example 3 | | | | | | |
| 4HR + HA + | 100% | 98% | 97% | 97% | 94% | 89% |
| HPMC(15%)-Example 4 | | | | | | |
| 4HR + HA + | 100% | 50% | 30% | 20% | 10% | 5% |
| water-Comparative | | | | | | |
| Example 1 | | | | | | |
| 4HR + HA + | 100% | 70% | 55% | 40% | 30% | 10% |
| gelatin(6%)- | | | | | | |
| Comparative | | | | | | |
| Example 2 | | | | | | |
| 4HR + HA + | 100% | 80% | 65% | 50% | 40% | 20% |
| gelatin(10%)- | | | | | | |
| Comparative | | | | | | |
| Example 3 | | | | | | |

In case of the bone graft materials of Comparative Examples 1 to 3 which do not contain HPMC, it is confirmed that there are problems in expecting consistent treatment effects due to 4-hexylresorcinol since moisture contents of the bone graft materials of Comparative Examples 1 to 3 are radically decreased as time goes on.

As described above, it will be understood that the aforementioned technical configuration of the present invention can be implemented in the form of other specific form by those skilled in the art to which the present invention pertains without changing technical ideas or essential features of the present invention.

Accordingly, the embodiments described above are exemplary in all aspects and are understood not to be limited, the scope of the present invention is shown by patent claims described later rather than the above-described specific description, and all changes or modified forms derived from meaning and scope of the patent claims and equivalent concepts thereof should be construed to be included in the scope of the present invention.

What is claimed is:

1. A bone graft material grafted with 4-hexylresorcinol affecting bone formation characterized in that it is produced by mixing hydrous ethanol and a base material for release control with distilled water or a salt-dissolved aqueous solution to prepare a mixed solution and precipitating a bone graft material into the mixed solution, thereby injecting 4-hexylresorcinol into the bone graft material, wherein 0.001 to 0.03 part by weight of 4-hexylresorcinol, 0.003 to 0.7 part by weight of the bone graft material, and 5 to 20 parts by weight of the base material for release control are mixed with respect to 100 parts by weight of the hydrous ethanol containing 10 to 60 vol % of water, the bone graft material includes one or more selected from hydroxyapatite, tricalcium phosphate, monocalcium phosphate, tetracalcium phosphate, and dicalcium phosphate, and the base material for release control includes one or more selected from hydroxypropyl methylcellulose (HPMC), methyl cellulose (MC), hydroxyethyl methyl cellulose (HEMC), ethyl cellulose (EC), carboxymethyl cellulose (CMC), collagen, and hyaluronic acid.
